(19) United States
(12) Patent Application Publication
(10) Pub. No.: US 2024/0237620 A1
(43) Pub. Date: Jul. 18, 2024

(54) METHOD AND APPARATUS FOR PREDICTING DEMANDED NET ENERGY FOR MAINTENANCE, AND ELECTRONIC DEVICE

(71) Applicant: CHINA AGRICULTURAL UNIVERSITY, Beijing (CN)

(72) Inventors: Shuai ZHANG, Beijing (CN); Zhe LI, Beijing (CN); Zhengcheng ZENG, Beijing (CN); Changhua LAI, Beijing (CN); Fenglai WANG, Beijing (CN)

(73) Assignee: CHINA AGRICULTURAL UNIVERSITY, Beijing (CN)

(21) Appl. No.: 18/561,869

(22) PCT Filed: Nov. 9, 2022
(86) PCT No.: PCT/CN2022/130843

§ 371 Date: Nov. 17, 2023

(30) Foreign Application Priority Data
Apr. 13, 2022 (CN) 202210388905.5

(51) Int. Cl. A01K 29/00; A61D 99/00; G16H 50/30

(52) U.S. Cl. CPC A01K 29/005; A61D 99/00; G16H 50/30

(57) ABSTRACT

Provided are a method and an apparatus for predicting a demanded net energy for maintenance, and an electronic device. The method comprises: acquiring a heart rate data of a target pig (110); and on the basis of the heart rate data and a pre-trained net energy demand prediction model, acquiring a demanded net energy for maintenance of the target pig, wherein the net energy demand prediction model is a neural network model acquired by a productivity parameter-based training (120). According to the method, a prediction based on the heart rate data of a pig is effectively introduced in the prediction process of the demanded net energy for maintenance of the pig. Moreover, the overall prediction process is simplified and optimized, so as to conveniently and quickly predict the demanded net energy for maintenance of the pig in real-time with improved reproducibility and applicability.

### CROSS REFERENCE OF RELATED APPLICATION

This application claims priority to Chinese Patent Application No. 202210388905.5, titled “METHOD AND APPARATUS FOR PREDICTING DEMANDED NET ENERGY FOR MAINTENANCE, AND ELECTRONIC DEVICE”, filed on Apr. 13, 2022 with the China National Intellectual Property Administration, which is incorporated herein by reference in its entirety.

### FIELD

The present disclosure relates to the technical field of feeding and management for swine, and in particular to a method and an apparatus for predicting net energy requirements for maintenance, and an electronic device.

### BACKGROUND

Energy systems involved in production and breeding industry of swine generally include the general energy system, the digestible energy system, the metabolizable energy system and the net energy system. Those four energy systems have ascending accuracies in describing energy available to swine. That is, the net energy system is a precise system for describing energy available to swine. In the net energy system, net energy requirements usually include net energy requirements for maintenance, net energy requirements for deposition, net energy requirements for protein deposition and net energy requirements for fat deposition. In a state of net energy requirements for maintenance, a swine is required to maintain basic metabolism, and an energy flow in the body of the swine is in dynamic balance without energy deposition and energy decomposition. In this case, net energy requirements only include net energy requirements for maintenance. Net energy requirements for maintenance represent a prerequisite for ensuring healthy growth and smooth parturition of swine. Therefore, it is of great significance in production and breeding industry of swine to effectively determine net energy requirements for maintenance of swine. Estimating net energy requirements for maintenance of swine is based on basic metabolic heat production of the swine, and the basic metabolic heat production is generally replaced by fasting metabolic heat production.

Indirect respiratory calorimetry is a conventional mainstream method for determining net energy requirements for maintenance of swine. In this method, each time net energy requirements for maintenance of a swine is to be determined, it is required to analyze compositions of gas inhaled and exhaled by the swine by using a dedicated respiratory calorimetry device. Fasting metabolic heat production of the swine in unit time is calculated based on a difference between the compositions of the gas inhaled and exhaled by the swine, and then the net energy requirements for maintenance of the swine is calculated based on the obtained fasting metabolic heat production.

However, the conventional method for determining net energy requirements for maintenance of swine using indirect respiratory calorimetry has a complex process, which results in poor reproducibility, so that the method is difficult to be popularized and applied in practical production.

### SUMMARY

A method and an apparatus for predicting net energy requirements for maintenance, and an electronic device are provided according to the present disclosure, to solve the problems of poor reproducibility and poor applicability that are caused by complexity of the conventional determination method in the conventional technology, so as to optimize a process of predicting net energy requirements for maintenance of swine, thereby improving the applicability of the prediction process.

A method for predicting net energy requirements for maintenance is provided according to the present disclosure. The method includes: obtaining heart rate data of a target swine; and obtaining net energy requirements for maintenance of the target swine based on the heart rate data and a pre-trained net-energy-requirements prediction model, where the net-energy-requirements prediction model is a neural network model obtained by training based on capacity parameters.

According to the method for predicting net energy requirements for maintenance provided in the present disclosure, a process of training the net-energy-requirements prediction model includes: obtaining heart rate data of several sample swine to form a first data set, where the heart rate data includes time information: obtaining net-energy-requirements-for-maintenance data of the several sample swine corresponding to the time information to form a second data set: establishing a training data set based on the first data set and the second data set, obtaining the capacity parameters based on the training data set, a preset data curve fitting approach and a preset parameter estimation algorithm: and training with respect to the capacity parameters based on the training data set to obtain the net-energy-requirements prediction model, where the data curve fitting approach is to perform curve fitting on the training data set based on a nonlinear logical regression function.

According to the method for predicting net energy requirements for maintenance provided in the present disclosure, the obtaining the capacity parameters based on the training data set, a preset data curve fitting approach and a preset parameter estimation algorithm includes: performing, by taking the heart rate data in the training data set as input and taking the net-energy-requirements-for-maintenance data, corresponding to the heart rate data, in the training data set as output, curve fitting based on a nonlinear logical regression function, to obtain a curve fitting function: and performing inverse estimation of parameters based on the preset parameter estimation algorithm and the curve fitting function to obtain the capacity parameters.

According to the method for predicting net energy requirements for maintenance provided in the present disclosure, the curve fitting function is expressed as:

$${NEm}_{ij} = g ( {Φ}_{i} , {HR}_{ij} ) + {ε}_{ij}$$

where i represents a serial number of a sample swine, j represents a serial number of data. HRᵢⱼ represents heart rate data of a sample swine, NEmᵢⱼ represents net energy requirements for maintenance of a sample swine at corresponding time, Φᵢ represents capacity parameters, and εᵢⱼ represents a random effect error.

According to the method for predicting net energy requirements for maintenance provided in the present disclosure, the parameter estimation algorithm includes any one or more of expectation-maximization algorithm, a Newton's iteration algorithm and a gradient descent algorithm.

According to the method for predicting net energy requirements for maintenance provided in the present disclosure, the process of training the net-energy-requirements prediction model further includes: establishing a test data set based on the first data set and the second data set, and recording the net-energy-requirements-for-maintenance data in the test data set as actual values of the net energy requirements for maintenance; inputting the heart rate data in the test data set to the net-energy-requirements prediction model to obtain predicted values of the net energy requirements for maintenance: analyzing correlation between the predicted values of the net energy requirements for maintenance and the actual values of the net energy requirements for maintenance: and verifying the net-energy-requirements prediction model based on the correlation.

According to the method for predicting net energy requirements for maintenance provided in the present disclosure, the verifying the net-energy-requirements prediction model based on the correlation includes: obtaining distribution of prediction weighted residuals based on the correlation: and verifying the net-energy-requirements prediction model based on the distribution of the prediction weighted residuals.

An apparatus for predicting net energy requirements for maintenance is further provided according to the present disclosure. The device includes an obtaining module and a prediction module. The obtaining module is configured to obtain heart rate data of a target swine. The prediction module is configured to obtain net energy requirements for maintenance of the target swine based on the heart rate data and a pre-trained net-energy-requirements prediction model, where the net-energy-requirements prediction model is a neural network model obtained by training based on capacity parameters.

An electronic device is further provided according to the present disclosure. The electronic device includes a processor and a memory storing a computer program executable by the processor. The processor is configured to execute the program to implement the method for predicting net energy requirements for maintenance according to any one of the above.

A non-transient computer-readable storage medium storing a computer program thereon is further provided according to the present disclosure. The computer program is configured to, when being executed by a processor, implement the method for predicting net energy requirements for maintenance according to any one of the above.

A method and an apparatus for predicting net energy requirements for maintenance, and an electronic device are provided according to the present disclosure. The method includes: obtaining heart rate data of a target swine: and obtaining net energy requirements for maintenance of the target swine based on the heart rate data and a pre-trained net-energy-requirements prediction model, where the net-energy-requirements prediction model is a neural network model obtained by training based on capacity parameters. In the method, an idea of predicting net energy requirements for maintenance of a swine based on heart rate data of the swine is effectively introduced, which simplifies and optimizes an overall prediction process, so as to conveniently and quickly predict the net energy requirements for maintenance of the swine in real time, thereby improving reproducibility and applicability of the method.

### BRIEF DESCRIPTION OF THE DRAWINGS

In order to more clearly describe the technical solutions in the present disclosure or the technical solutions in the conventional technology, drawings to be used in the description of the embodiments or the conventional technology are briefly described hereinafter. It is apparent that the drawings described below are merely used for describing the embodiments of the present disclosure, and those skilled in the art may obtain other drawings according to the provided drawings without any creative effort.

FIG. 1 is a flowchart of a method for predicting net energy requirements for maintenance according to the present disclosure;

FIG. 2 is a first schematic diagram of a process of training a net-energy-requirements prediction model in the method for predicting net energy requirements for maintenance according to the present disclosure:

FIG. 3 is a second schematic diagram of the process of training the net-energy-requirements prediction model in the method for predicting net energy requirements for maintenance according to the present disclosure;

FIG. 4 is a third schematic diagram of the process of training the net-energy-requirements prediction model in the method for predicting net energy requirements for maintenance according to the present disclosure:

FIG. 5 is a fourth schematic diagram of the process of training the net-energy-requirements prediction model in the method for predicting net energy requirements for maintenance according to the present disclosure;

FIG. 6 is a schematic diagram showing data curve fitting of heart rate data and net-energy-requirements-for-maintenance data of any pregnant sow in the method for predicting net energy requirements for maintenance according to the present disclosure;

FIG. 7 is a schematic diagram showing correlation between predicted values of net energy requirements for maintenance and actual values of the net energy requirements for maintenance in the method for predicting net energy requirements for maintenance according to the present disclosure;

FIG. 8 is a schematic diagram showing distribution of prediction weighted residuals of a net-energy-requirements prediction model in different heart rate ranges in the method for predicting net energy requirements for maintenance according to the present disclosure;

FIG. 9 is a schematic diagram showing distribution of the prediction weighted residuals of the net-energy-requirements prediction model in different net-energy-requirements-for-maintenance ranges in the method for predicting net energy requirements for maintenance according to the present disclosure;

FIG. 10 is a schematic structural diagram of an apparatus for predicting net energy requirements for maintenance according to the present disclosure; and

FIG. 11 is a schematic structural diagram of an electronic device according to the present disclosure.

Reference numbers in the drawings:

| | |
|---|---|
| 101: obtaining module | 102: prediction module |
| 1110: processor | 1120: communications interface |
| 1130: memory | 1140: communication bus |

### DETAILED DESCRIPTION OF EMBODIMENTS

In order to make the objective, the technical solutions and advantages of the present disclosure clear, technical solutions of the present disclosure are described below clearly and completely in conjunction with the drawings of the present disclosure. Apparently, the embodiments described below are only some embodiments rather than all the embodiments of the present disclosure. Any other embodiments obtained by those skilled in the art based on the embodiments in the present disclosure without any creative effort fall within the protection scope of the present disclosure.

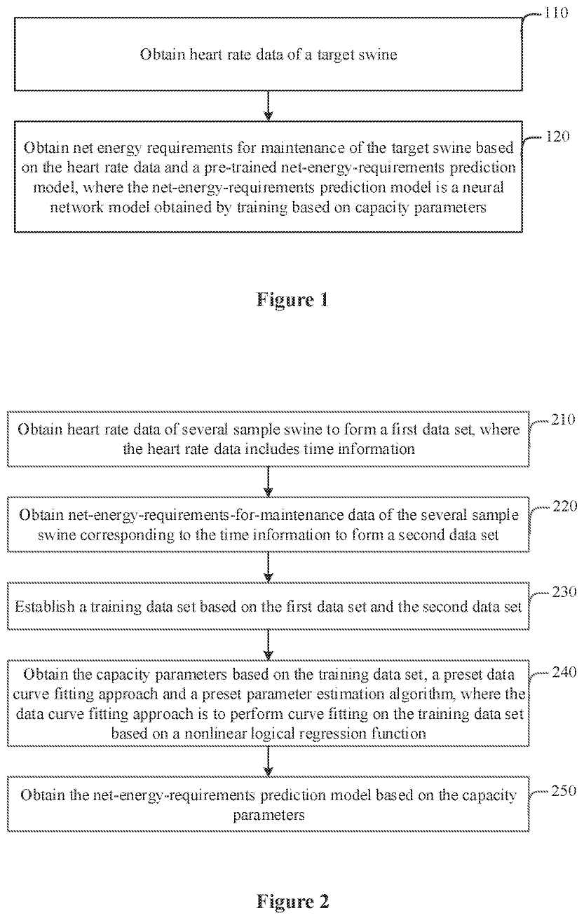

A method and an apparatus for predicting net energy requirements for maintenance, and an electronic device according to the present disclosure are described below in conjunction with FIG. 1 to FIG. 11.

Almost all the oxygen required for energy metabolism and heat production in an animal body comes from blood circulation in the animal body, and there is a significant linear correlation between heart rates of the animal and oxygen consumption of the animal. In calculation of heat production of an animal, oxygen consumption of the animal is usually the most important parameters. Based on the significant linear correlation, it can be concluded that heat production of an animal can be calculated based on heart rates of the animal. That is, oxygen consumption and heat production of an animal can be effectively predicted based on heart rates of the animal, and thus net energy requirements for maintenance of the animal can be predicted based on the heat production of the animal. The method of predicting body energy consumption based on heart rates has been applied for humans, but it is rarely applied in swine.

With the method according to the present disclosure, net energy requirements for maintenance of swine at various physiological stages such as pregnant sows, lactating sows, piglets, fattening pigs and breeding boars can be efficiently predicted in real time. The embodiments of the present disclosure are illustrated by taking pregnant sows as an example.

A method for predicting net energy requirements for maintenance is provided according to the present disclosure. FIG. 1 is a flowchart of the method for predicting net energy requirements for maintenance according to the present disclosure. As shown in FIG. 1, the method includes the following step 110 and step 120.

In step 110, heart rate data of a target swine is obtained.

Heart rate data of a target swine under a normal feeding condition is obtained by using a heart rate measuring instrument such as a heart rate sensor.

In step 120, net energy requirements for maintenance of the target swine is obtained based on the heart rate data and a pre-trained net-energy-requirements prediction model. The net-energy-requirements prediction model is a neural network model obtained by training based on capacity parameters.

The heart rate data of the target swine under the normal feeding condition obtained in step 110 is inputted to the pre-trained net-energy-requirements prediction model, and the net-energy-requirements prediction model outputs the net energy requirements for maintenance of the target swine under the normal feeding condition. The net-energy-requirements prediction model is a neural network model obtained by training with the capacity parameters.

A method for predicting net energy requirements for maintenance is provided according to the present disclosure. With the method, heart rate data of a target swine is obtained, and net energy requirements for maintenance of the target swine is obtained based on the heart rate data and a pre-trained net-energy-requirements prediction model. The net-energy-requirements prediction model is a neural network model obtained by training based on capacity parameters. In the method, an idea of predicting net energy requirements for maintenance of a swine based on heart rate data of the swine is effectively introduced, which simplifies and optimizes an overall prediction process, so as to conveniently and quickly predict the net energy requirements for maintenance of the swine in a maintenance state in real time, thereby improving reproducibility and applicability of the method and promoting the application of the method in feeding, management, and production for swine.

According to the method for predicting net energy requirements for maintenance provided in the present disclosure, FIG. 2 is a first schematic diagram of a process of training the net-energy-requirements prediction model in the method for predicting net energy requirements for maintenance according to the present disclosure. As shown in FIG. 2, the process of training the net-energy-requirements prediction model includes the following step 210 to step 250.

In step 210, heart rate data of several sample swine is obtained to form a first data set. The heart rate data includes time information.

The heart rate data of multiple sample swine are collected by using a heart rate collecting device (e.g., a heart rate monitor), to form the first data set. The heart rate data includes time information. The time information refers to information indicating a time instant at which the heart rate data is collected or indicating a time period during which the heart rate data is collected. The time information is used to realize one-to-one correspondence between heart rate data and net energy requirements for maintenance of a same sample swine through correspondence of time instants or time periods. In a case that the time information is information indicating a time instant (also referred to as time stamps), the collected heart rate data of the sample swine includes real-time heart rates. In a case that the time information is information indicating a time period, the collected heart rate data of the sample swine includes an average of heart rates in the time period.

In step 220, net-energy-requirements-for-maintenance data of the several sample swine corresponding to the time information is obtained, to form a second data set.

The net-energy-requirements-for-maintenance data of the several sample swine is obtained from fasting metabolic heat production determined with indirect respiratory calorimetry. Respiratory calorimetry experiment is performed on the sample swine in a fasting state to determine O₂ consumptions by the sample swine and CO₂ and CH₄ emissions from the sample swine, fasting heat production per unit of metabolic weight is calculated for the sample swine, whereby the net-energy-requirements-for-maintenance data is obtained through estimation, so as to form the second data set.

In step 230, a training data set is formed based on the first data set and the second data set.

A preset proportion of data is selected from the first data set as training data, and the same preset proportion of corresponding data is accordingly selected from the second data set as training data. The training data from the first data set and the training data from the second training set form the training data set.

In step 240, the capacity parameters are obtained based on the training data set, a preset data curve fitting approach and a preset parameter estimation algorithm. In the data curve fitting approach, curve fitting is performed on the training data set based on a nonlinear logical regression function.

Data curve fitting is performed on input data and output data among the training data in the training data set with the preset data curve fitting approach, and the capacity parameters are calculated based on a data curve fitting result and the preset parameter estimation algorithm.

It should be noted that, there may be multiple types of capacity parameters, which depends on practical applications, and the multiple types of capacity parameters are all to be calculated.

In step 250, training with respect to the capacity parameters is performed based on the training data set to obtain the net-energy-requirements prediction model.

Based on the one or more types of capacity parameters obtained in step 240, it may be regarded as performing in-depth learning and training with respect to the obtained one or more types of capacity parameters by using the training data set and the nonlinear logistic regression function, so as to establish and train the net-energy-requirements prediction model.

The trained net energy requirements prediction model can be effectively applied to the process of predicting net energy requirements for maintenance of other target swine.

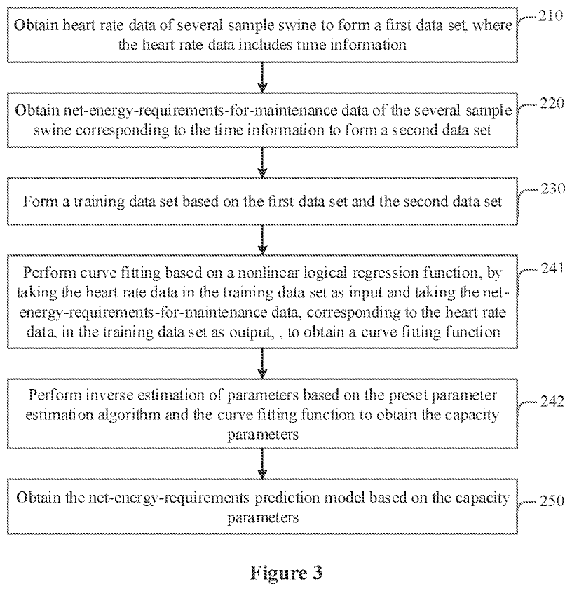

According to the method for predicting net energy requirements for maintenance provided in the present disclosure. FIG. 3 is a second schematic diagram of the process of training the net-energy-requirements prediction model in the method for predicting net energy requirements for maintenance according to the present disclosure. As shown in FIG. 3, based on FIG. 2, the step 240, in which the capacity parameters are obtained based on the training data set, the preset data curve fitting approach and the preset parameter estimation algorithm, includes the following step 241 and step 242.

In step 241, curve fitting is performed based on the nonlinear logical regression function, by taking the heart rate data in the training data set as input and taking the net-energy-requirements-for-maintenance data, corresponding to the heart rate data, in the training data set as output, to obtain a curve fitting function.

Curve fitting is performed based on a logistic regression function in a nonlinear mixed model, by taking the heart rate data in the training data set as input and taking the net-energy-requirements-for-maintenance data, corresponding to the heart rate data, in the training data set as output, on the heart rate data and the corresponding net-energy-requirements-for-maintenance data of the sample swine in the training data set. Sample swine individuals are randomly selected, and finally the curve fitting function is obtained.

In step 242, inverse estimation of parameters is performed based on the preset parameter estimation algorithm and the curve fitting function to obtain the capacity parameters.

Inverse estimation of parameters is performed on the curve fitting function based on the preset parameter estimation algorithm, and thus parameters in the curve fitting function, i.e., the capacity parameters, are calculated.

According to the method for predicting net energy requirements for maintenance provided in the present disclosure, the curve fitting function is expressed as:

$${NEm}_{ij} = g ( {Φ}_{i} , {HR}_{ij} ) + {ε}_{ij}$$

In the above equation, i represents a serial number of sample swine, j represents a serial number of data. HRᵢⱼ represents heart rate data of a sample swine, NEmᵢⱼ represents net energy requirements for maintenance of a sample swine at corresponding time, Φᵢ represents capacity parameters, and εᵢⱼ represents a random effect error.

In addition:

$$g ( {Φ}_{i} , {HR}_{ij} ) = \frac{{ϕ}_{1 i}}{1 + exp [ \frac{{ϕ}_{2 i} - {HR}_{ij}}{{ϕ}_{3 i}} ]}$$

In the above equation, Φᵢ=(ϕ₁ᵢ, ϕ₂ᵢ, ϕ₃ᵢ) represents capacity parameters, or model parameters. In this case, there are three model parameters, namely ϕ₁ᵢ, ϕ₂ᵢ, ϕ₃ᵢ. That is, one type of capacity parameters Φᵢ includes three capacity parameters.

According to the method for predicting net energy requirements for maintenance provided in the present disclosure, the parameter estimation algorithm includes any one or more of expectation-maximization algorithm, Newton's iteration algorithm and gradient descent algorithm.

The preset parameter estimation algorithm may be any one or a combination of the expectation-maximization algorithm, the Newton's iteration algorithm and the gradient descent algorithm. The expectation-maximization algorithm refers to Stochastic approximation expectation maximization (SAEM) algorithm. The accuracy of parameter estimation can be significantly improved by performing inverse estimation of parameters based on the above algorithms.

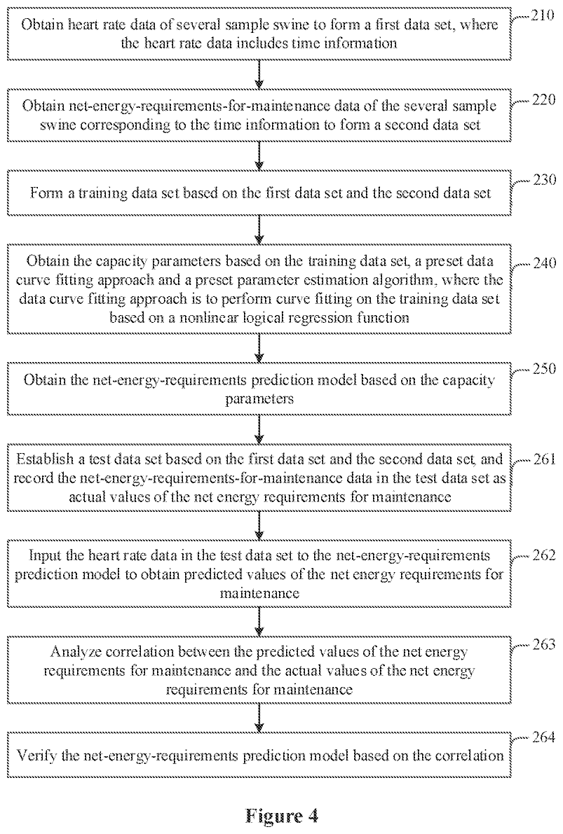

According to the method for predicting net energy requirements for maintenance provided in the present disclosure, FIG. 4 is a third schematic diagram of the process of training the net-energy-requirements prediction model in the method for predicting net energy requirements for maintenance according to the present disclosure. As shown in FIG. 4, based on FIG. 2, the process of training the net-energy-requirements prediction model further includes the following step 261 to step 264.

In step 261, a test data set is established based on the first data set and the second data set, and the net-energy-requirements-for-maintenance data in the test data set is recorded as actual values of the net energy requirements for maintenance.

Heart rate data other than training data is selected from the first data set as test data according to a preset proportion, and net-energy-requirements-for-maintenance data other than training data is accordingly selected from the second data set as test data according to the same preset proportion. Further, the test data from the first data set and the test data from the second training set form the test data set. The net-energy-requirements-for-maintenance data, in the test data set is recorded as the actual values of the net energy requirements for maintenance.

In step 262, the heart rate data in the test data set is inputted to the net-energy-requirements prediction model to obtain predicted values of the net energy requirements for maintenance.

The net-energy-requirements prediction model is established and trained through the above step 210 to step 250. The model does not have fixed prediction effect and is required to be verified. The heart rate data in the test data set is inputted to the net-energy-requirements prediction model to obtain predicted values of the net energy requirements for maintenance of the swine at corresponding time.

In step 263, correlation between the predicted values of the net energy requirements for maintenance and the actual values of the net energy requirements for maintenance is analyzed.

The correlation between the actual values of the net energy requirements for maintenance obtained in step 262 and the predicted values of the net energy requirements for maintenance obtained in step 263 is analyzed. A small difference between the actual values and the predicted values indicates strong correlation, and a large difference between the actual values and the predicted values indicates weak correlation.

In step 264, the net-energy-requirements prediction model is verified based on the correlation.

The prediction effect of the net-energy-requirements prediction model is verified based on strength of the correlation. Strong correlation indicates that the predicted values of the net energy requirements for maintenance described in step 263 are close to the actual values of the net energy requirements for maintenance described in step 262, and indicates a small error and a good prediction effect of the net-energy-requirements prediction model. Weak correlation indicates a poor prediction effect of the net-energy-requirements prediction model. In a case that the net-energy-requirements prediction model has a poor prediction effect, the net-energy-requirements prediction model may be further optimized based on the verification result.

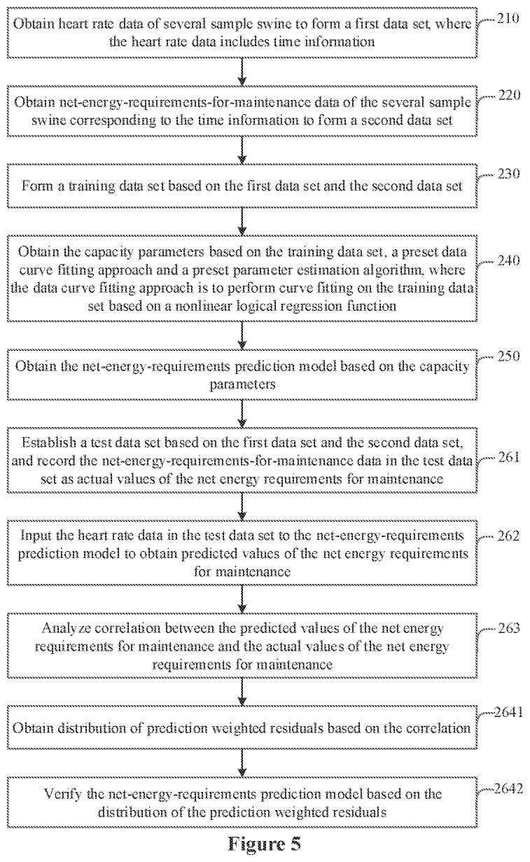

According to the method for predicting net energy requirements for maintenance provided in the present disclosure. FIG. 5 is a fourth schematic diagram of the process of training the net-energy-requirements prediction model in the method for predicting net energy requirements for maintenance according to the present disclosure. As shown in FIG. 5, based on FIG. 4, the step 264, in which the net-energy-requirements prediction model is verified based on the correlation, includes the following step 2641 and step 2642.

In step 2641, distribution of prediction weighted residuals is obtained based on the correlation.

Distribution of prediction weighted residuals of prediction results, including distribution of the prediction weighted residuals based on different heart rate ranges and distribution of the prediction weighted residuals based on different net-energy-requirements-for-maintenance ranges, may further be obtained based on the correlation between the actual values of the net energy requirements for maintenance described in step 262 and the predicted values of the net energy requirements for maintenance described in step 263.

In step 2642, the net-energy-requirements prediction model is verified based on the distribution of the prediction weighted residuals.

The prediction effect of the net-energy-requirements prediction model is further analyzed based on the distribution of the prediction weighted residuals in different rate data ranges, or based on the distribution of the prediction weighted residuals in different net-energy-requirements-for-maintenance ranges.

Implementations are described as follows by taking pregnant sows as an example.

In step (1), heart rate data of several sample swine (pregnant sows as samples) are obtained to form a first data set. The heart rate data includes time information.

Dedicated heart rate sensors based on electrocardiogram (ECG) signals are put on the pregnant sows, and the heart rate data of the pregnant sows are obtained by using the dedicated heart rate sensors.

Monitoring and prediction are performed in an animal experiment center in Hebei Province of China to ensure the preciseness of the process and data.

Six Landrace*LargeWhite binary hybrid multiparous pregnant sows are selected as to-be-monitored objects. For each of the pregnant sows, pregnancy time is 69 days (d69) and an initial weight is within the range of (232.5±12.5)kg. The pregnant sows are fed separately in respective dedicated metabolic cages (1.70 m*0.70 m*1.40 m). During the whole process, the pregnant sows are fed twice each day at fixed time instants (8:30 and 15:30 each day), and the pregnant sows are always allowed to drink water freely. Daily ration for feeding is a standard corn and soybean meal ration, to meet nutritional requirements of the pregnant sows. Formula composition and nutritional level follow basic feeding formula and percentage.

Open circulating respiratory calorimetry devices (referred to as respiratory calorimeters) dedicated for swine are set. For each of the respiratory calorimeters, a temperature is controlled within the range of (20±1° C.), humidity is controlled at about 70%, a wind speed in the space is controlled at about 1 m/s, and a fixed light exposure condition is set (light exposure time is set from 06:00 to 18:00). In addition to time spent on feeding and collecting feces and urine, the whole process lasts for 9 days. All pregnant sows are controlled to stay in their respective metabolic cages for 5 days for adaption, and then the pregnant sows are transferred their respective respiratory calorimeters (each pregnant sow has a separate respiratory calorimeter). In addition, the pregnant sows are controlled to start fasting at 18:00 on the 8th day. For each of the pregnant sows, a heart rate of the pregnant sow at a time instant within 24 hours after the pregnant sow start fasting, and fasting metabolic heat production of the pregnant sow at the corresponding time instant (or, an average of heart rates of the pregnant sow during a time period within 24 hours after the pregnant sow start fasting, and an average of fasting metabolic heat productions of the pregnant sow during the corresponding time period) are recorded.

The pregnant sows wear portable electronic heart rate monitors before being transferred to the respiratory calorimeters. The heart rate monitor is a Polar H10 heart rate monitor (including a heart rate sensor and an elastic electrode strip). The heart rate monitor is based on the principle of ECG signals. The heart rate sensor transmits ECG signals through Bluetooth and ANT^{+™} technology. The heart rate monitor is paired with a receiving device such as a smart phone. In usage, electrode points on the elastic electrode strip are wetted, and then the heart rate monitor is bound on the pregnant sow at a part on the chest close to an inner side of forelegs, to ensure that the electrodes fully contact skin and subcutaneous tissue with dense blood vessels of the pregnant sow. After the heart rate monitors are successfully paired with a smart phone, a Polar account may be created using the smart phone, and it starts to measure heart rates of the six pregnant sows at certain time instants, or averages of heart rates during a certain time period for each pregnant sow. All obtained heart rate data form a first data set. Moreover, the heart rate data includes time information. The time information refers to information indicating the time instant at which heart rates are acquired or the time period during which heart rates are acquired.

The Polar H10 heart rate monitor has a function of storing data. After the measurement, the obtained heart rate data may be synchronized on the Internet by accessing the Polar account, and then the heart rate data is downloaded and saved in a CSV/TCX file format for subsequent analysis.

(2) Net-energy-requirements-for-maintenance data of the six pregnant sows corresponding to the time information is obtained to form a second data set.

Fasting metabolic heat productions are determined based on indirect respiratory calorimetry to obtain the net-energy-requirements-for-maintenance data of the respective pregnant sows. Respiratory calorimetry experiment is performed on the pregnant sows in a fasting state to determine O₂ consumptions by the pregnant sows and CO₂ and CH₄ emissions from the pregnant sows, fasting heat productions of the pregnant sows per unit of metabolic weight are calculated, and whereby the net-energy-requirements-for-maintenance data is obtained through estimation.

Fasting metabolic heat production data of the pregnant sows is obtained by using the open circulating respiratory calorimeters. The pregnant sows adapt to the daily ration and the environment of the separate metabolic cages from the 1st day to the 4th day. On the 5th day, environments in the respiratory calorimeters are set, all pregnant sows are transferred to respective respiratory calorimeters, and the heart rate monitors are calibrated to be in states the same as the states in the above step (1). The pregnant sows are controlled to start fasting for 24 hours from 18:00 on the 8th day, and for each pregnant sow, fasting metabolic heat productions of the pregnant sow at time instants for measuring the heart rate data (or averages of fasting metabolic heat productions of the pregnant sow during time period of measuring the heart rate data) is measured.

The fasting metabolic heat productions are measured as follows. For gas compositions, O₂ is measured by a paramagnetic oxygen analyzer (Oxymat 6E, Siemens/Munich, produced in Germany), CO₂, NH₃ and CH₄ are measured by an infrared analyzer (Ultramat 6E, Siemens/Munich, produced in Germany), and an exhaust flow is measured by a gas mass flowmeter (Alicat/Tucson, produced in America). A total of six respiratory calorimeters are used to measure the fasting metabolic heat productions of the six pregnant sows. Each two respiratory calorimeters share a same set of gas analysis system and each respiratory calorimeter performs gas measurement every 5 minutes. The fasting metabolic heat productions are calculated through gas analysis and thus the net energy requirements for maintenance of the pregnant sows is calculated, which is describe as follows.

In step (2-1), a volume of exhaled gas is converted to a standard volume in a standard state (0° C., 1013 hPa).

A volume V of exhaled gas during each time period is calculated according to V=time(min)*flow rate(L/min) of gas.

A standard volume SV of the exhaled gas is calculated according to:

$$SV = V * ( P - Pw ) / 1013 * 273 / ( 273 + T ) ; and$$

$${P}_{w} = RH / 100 * ( 3.999 + 0.45547 T + 0.001708 {T}^{2} + 0.000469 {T}^{3} ) ,$$

where SV represents standard volume (in a state of 0° C. and 1013 hPa) of the exhaled gas, V represents actual volume of exhaled gas. P represents air pressure in the respiratory calorimeter; P_{w} represents vapor pressure; T represents temperature in the respiratory calorimeter; and RH represents relative humidity in the respiratory calorimeter.

In step (2-2), after the volume of the exhaled gas is converted to the standard volume, for each pregnant sow, a volume of O₂ inhaled by the pregnant sow and a volume of CO₂ exhaled by the pregnant sow during the experiment are calculated according to:

$${V}_{{CO}_{2} , production} = ( {C}_{{CO}_{2} , b} - {C}_{{CO}_{2} , a} ) * {SV}_{respiratory calorimeter} + ( {C}_{{CO}_{2} , a} - {C}_{{CO}_{2} , air} ) * {SV}_{exhaled} ; and$$

$${V}_{{O}_{2} , production} = ( {C}_{{O}_{2} , a} - {C}_{{O}_{2} , b} ) * {SV}_{respiratory calorimeter} + ( {C}_{{O}_{2} , a} - {C}_{{O}_{2} , air} ) * {SV}_{exhaled}$$

In the above equations, C_{CO}_{₂}_{,a} and C_{CO}_{₂}_{,b} represent concentrations (%) of CO₂ at the beginning of breathing and at the end of breathing respectively; C_{O}_{₂}_{,a} and C_{O}_{₂}_{,b} represent concentrations (%) of O₂ at the beginning of breathing and at the end of breathing respectively: SV_{respiratory calorimeter} represents a net usable standard volume of the respiratory calorimeter; and SV_{exhaled} represents a standard volume of the exhaled gas.

In step (2-3), for each pregnant sow, net energy requirements for maintenance of the pregnant sow is calculated according to the following equations, where the net energy requirements for maintenance is equal to the fasting metabolic heat production of the pregnant sow by default:
net energy requirements for maintenance (KJ/d/BW^{0.75})=fasting metabolic heat production (k.J/dBW^{0.75}); and
fasting metabolic heat production (kJ)=16.1753*O₂(L)+5.0208*CO₂(L)-2.1673*CH₄(L).

The second data set is formed based on the obtained net-energy-requirements-for-maintenance data of the pregnant sows.

In step (3), data standardization preprocessing is formed on the data obtained in step (1) and the data obtained in step (2) to obtain accurate and comprehensive first data set and second data set. Apparently, the data standardization preprocessing is optional and is only a preferred measure.

Data reorganization, and data screening and cleaning are performed on the data obtained in step (1) and the data obtained in step (2). The data reorganization includes abnormal data elimination, data completion and data value calculation. Data reorganization, and data screening and cleaning are performed on both of the heart rate data obtained in step (1) and the net-energy-requirements-for-maintenance data of the pregnant sows obtained in step (2). The heart rate data subjected to data standardization preprocessing and the net-energy-requirements-for-maintenance data subjected to data standardization preprocessing correspond to each other in terms of the time information. The heart rate data subjected to data standardization preprocessing and the net-energy-requirements-for-maintenance data subjected to data standardization preprocessing may be data in one-to-one correspondence in terms of time instants, or may be average data in one-to-one correspondence in terms of time periods. For example, for each pregnant sow, an average heart rate per hour of the pregnant sow and an average net-energy-requirements-for-maintenance of the pregnant sow in the corresponding hour are in one-to-one correspondence. One-to-one correspondence in terms of time information refers to that time at which the net-energy-requirements-for-maintenance data of the pregnant sows is obtained is consistent with time at which heart rate data of the pregnant sows is obtained, so as to form the one-to-one correspondence in terms of time information.

In step (4), a training data set is established based on the first data set and the second data set, and a test data set is established based on the first data set and the second data set.

The first data set is divided into training data and test data according to a preset proportion. Accordingly, the second data set is divided into training data and test data according to the same preset proportion. Further, the training data from the first data set and the training data from the second training set form the training data set. The test data from the first data set and the test data from the second training set form the test data set.

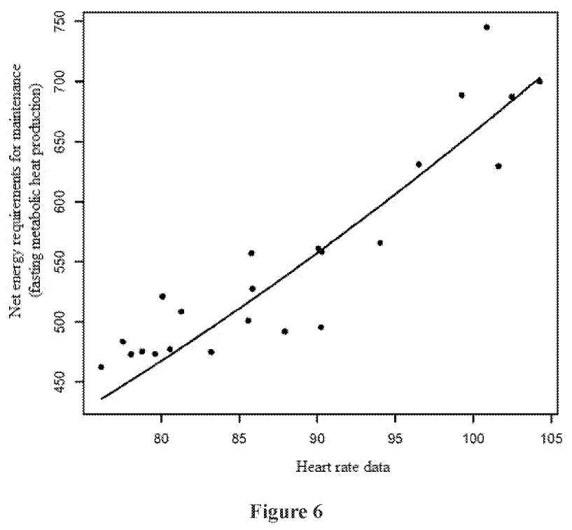

In step (5), curve fitting is performed based on a logistic regression function in a nonlinear mixed model, by taking the heart rate data in the training data set as input and taking the net-energy-requirements-for-maintenance data, corresponding to the heart rate data, in the training data set as output, on the heart rate data and the corresponding net-energy-requirements-for-maintenance data, of the pregnant sows in the training data set. Swine individuals are randomly selected. FIG. 6 is a schematic diagram showing data curve fitting of heart rate data and net-energy-requirements-for-maintenance data of any pregnant sow in the method for predicting net energy requirements for maintenance according to the present disclosure. For example, FIG. 6 shows a data curve fitting result of heart rate data and net-energy-requirements-for-maintenance data of a third pregnant sow. As shown in FIG. 6, the obtained curve fitting function is expressed as:

$${NEm}_{ij} = g ( {Φ}_{i} , {HR}_{ij} ) + {ε}_{ij}$$

In the above equation, i represents a serial number of sample swine, j represents a serial number of data, HRᵢⱼ represents heart rate data of a sample swine, NEmᵢⱼ represents net energy requirements for maintenance of a sample swine at corresponding time, Φᵢ represents capacity parameters, and εᵢⱼ represents a random effect error.

In addition:

$$g ( {Φ}_{i} , {HR}_{ij} ) = \frac{{ϕ}_{1 i}}{1 + exp [ \frac{{ϕ}_{2 i} - {HR}_{ij}}{{ϕ}_{3 i}} ]}$$

In the above equation, Φᵢ=(ϕ₁ᵢ, ϕ₂ᵢ, ϕ₃ᵢ) represents capacity parameters, or model parameters. In this case, there are three model parameters, namely ϕ₁ᵢ, ϕ₂ᵢ, ϕ₃ᵢ. That is, one type of capacity parameters Φᵢ includes three capacity parameters.

In step (6), inverse estimation of parameters is performed based on a preset parameter estimation algorithm and the curve fitting function to obtain the capacity parameters.

Inverse estimation of parameters is performed for Φᵢ=(ϕ₁ᵢ, ϕ₂ᵢ, ϕ₃ᵢ) in the above curve fitting function by taking the stochastic approximation expectation maximization (SAEM) algorithm as the preset parameter estimation algorithm, to obtain estimated values of three capacity parameters ϕ₁ᵢ, ϕ₂ᵢ, ϕ₃ᵢ.

For example, the estimated results of the parameters are shown in the following Table 1.

TABLE 1

| Capacity | Estimated value of | Standard | Coefficient of |
|---|---|---|---|
| parameter | capacity parameter | Error (SE) | variation (CV, %) |
| ϕ₁ | 1990 | 936.2 | 47.0 |
| ϕ₂ | 136 | 35.8 | 26.4 |
| ϕ₃ | 43 | 8.0 | 18.4 |

In step (7), training with respect to the capacity parameters is performed based on the training data set to obtain the net-energy-requirements prediction model.

An expression of the curve function is determined based on the estimated values of the capacity parameters, and training with respect to the capacity parameters is performed based on the training data set, so as to obtain the net-energy-requirements prediction model.

As mentioned above, the curve function is expressed as:

$${NEm}_{ij} = \frac{1990}{1 + exp [ \frac{136 - {HR}_{ij}}{43} ]} + {ε}_{ij}$$

Therefore, in a case that the random effect error εᵢⱼ can be ignored or is already determined, it is only required to obtain the heart rate data of a target swine under a normal feeding condition in advance and then substitute the heart rate data to HRᵢⱼ in the above equation, so as to quickly calculate the net energy requirements for maintenance NEmᵢⱼ of the target swine.

In step (8), the net-energy-requirements prediction model is verified based on the correlation between predicted values of the net energy requirements for maintenance and actual values of the net energy requirements for maintenance.

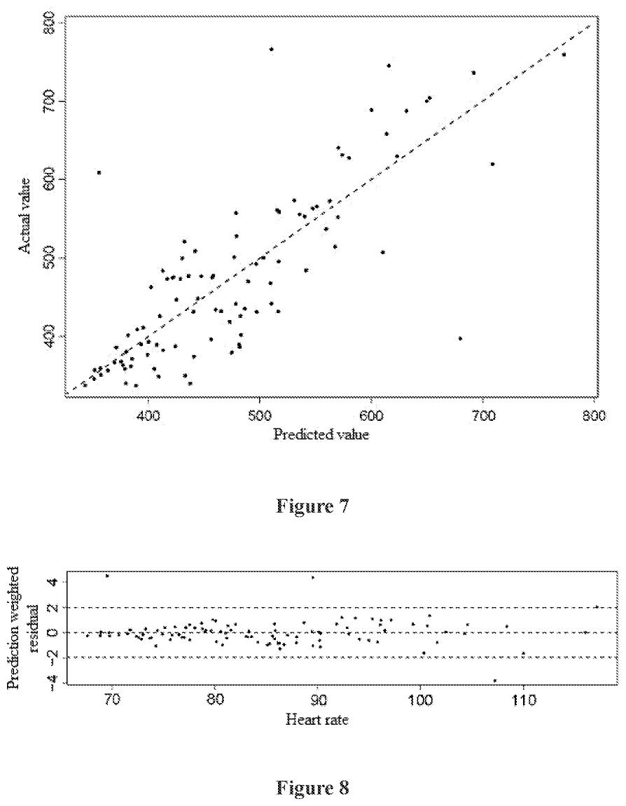

The net-energy-requirements-for-maintenance data in the test data set is recorded as the actual values of the net energy requirements for maintenance. The heart rate data in the test data set is inputted to the net-energy-requirements prediction model to obtain the predicted values of the net energy requirements for maintenance. The correlation between the predicted values of the net energy requirements for maintenance and the actual values of the net energy requirements for maintenance is analyzed, and a schematic diagram showing the correlation between the predicted values of the net energy requirements for maintenance and the actual values of the net energy requirements for maintenance as shown in FIG. 7 is drawn. Finally, the net-energy-requirements prediction model is verified based on the correlation. Specifically, by taking a pair of a predicted value and an actual vale as abscissa and ordinate of a coordinate point, multiple pairs of predicted values and actual values form multiple coordinate points shown in FIG. 7. A dotted line at 45 degrees in FIG. 7 represents a reference line. If the coordinate points are closer to the reference line, it indicates that the predicted values are closer to the actual values, i.e., stronger correlation between the predicted values and the actual values, and thus the net-energy-requirements prediction model is verified based on the correlation between the predicted values of the net energy requirements for maintenance and the actual values of the net energy requirements for maintenance shown in FIG. 7.

In the case of determining that the prediction effect of the net-energy-requirements prediction model is unsatisfactory, the net-energy-requirements prediction model may be further optimized based on a verification result in this case and relevant data to improve the prediction precision of the model.

(9) The net-energy-requirements prediction model is verified based on distribution of prediction weighted residuals.

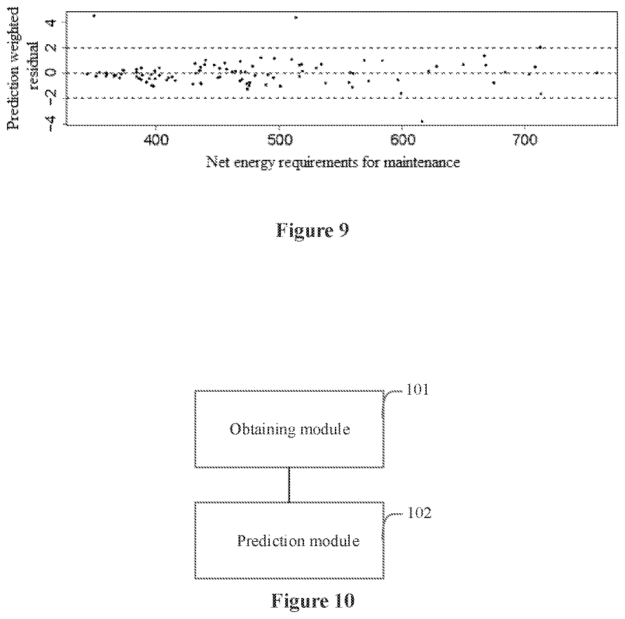

The distribution of the prediction weighted residuals is obtained based on the correlation between the predicted values of the net energy requirements for maintenance and the actual values of the net energy requirements for maintenance. For example, distribution of the prediction weighted residuals in different heart rate ranges as shown in FIG. 8 is obtained, and distribution of the prediction weighted residuals in different net-energy-requirements-for-maintenance ranges as shown in FIG. 9 is obtained. The net-energy-requirements prediction model is further verified based on the distribution of the prediction weighted residuals in different heart rate ranges or the distribution of the prediction weighted residuals in different net-energy-requirements-for-maintenance ranges. Specifically, if the prediction weighted residuals are distributed closer to the zero-value coordinate axis, or if the prediction weighted residuals fluctuate within a smaller range relative to the zero-value coordinate axis, it indicates that the model has better prediction effect, and otherwise the model has worse prediction effect.

In the case of determining that the prediction effect of the net-energy-requirements prediction model is poor, the net-energy-requirements prediction model may be further optimized based on a verification result in this case and relevant data, so as to improve the prediction precision of the model to a greater extent.

All the calculation in this embodiment is performed by using a saemix package in R 4.1.2 (R Core Team, 2022).

Compared with the conventional indirect respiratory calorimetry, the method for predicting net energy requirements for maintenance based on heart rates has little impact on a to-be-experimented object and has a simple and optimal process, and monitoring and prediction can be implemented easily with the method. The method provides a new solution for the study of the net energy system and energy requirements of swine. With the method, energy metabolism states of the pregnant sows can be monitored, and the net energy requirements for maintenance of the pregnant sows can be predicted conveniently and quickly, thus providing an accurate reference for a feeding and management strategy and feed formulation/adjustment for the pregnant sows and providing a basic theoretical support for development of an intelligent breeding internet of things monitoring device and a feeding device for pregnant sows. With the method, the expensive cost caused by the large respiratory calorimetry device required by the conventional method for predicting net energy requirements for maintenance of a sow can be effectively avoided, so as to reduce the cost.

An apparatus for predicting net energy requirements for maintenance is further provided according to the present disclosure. The method for predicting net energy requirements for maintenance and the apparatus for predicting net energy requirements for maintenance have same technical principles, and correspond to each other, which are not repeated here.

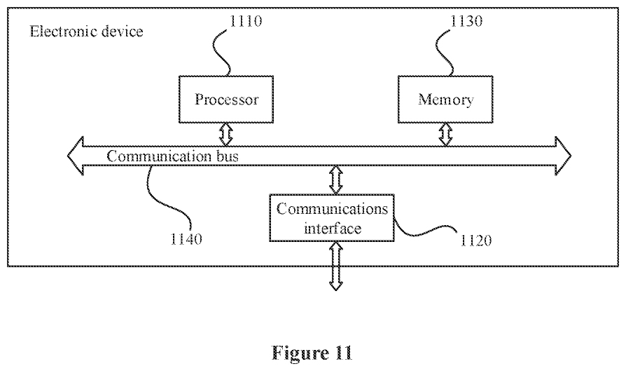

An apparatus for predicting net energy requirements for maintenance is further provided according to the present disclosure. FIG. 10 is a schematic structural diagram of an apparatus for predicting net energy requirements for maintenance according to the present disclosure. As shown in FIG. 10, the device includes an obtaining module 101 and a prediction module 102.

The obtaining module 101 is configured to obtain heart rate data of a target swine.

The prediction module 102 is configured to obtain net energy requirements for maintenance of the target swine based on the heart rate data and a pre-trained net-energy-requirements prediction model. The net-energy-requirements prediction model is a neural network model obtained by training based on capacity parameters.

An apparatus for predicting net energy requirements for maintenance is provided according to the present disclosure. The device includes an obtaining module 101 and a prediction module 102. The two modules cooperate with each other, thus the heart rate data of the target swine is obtained by the apparatus, and the net energy requirements for maintenance of the target swine is obtained based on the heart rate data and the pre-trained net-energy-requirements prediction model. The net-energy-requirements prediction model is a neural network model obtained by training based on capacity parameters. In the method, an idea of predicting net energy requirements for maintenance of a swine based on heart rate data of the swine is effectively introduced, so as to conveniently and quickly predict the net energy requirements for maintenance of the swine in a maintenance state in real time, thereby improving reproducibility and applicability of the device and promoting the application of the device in feeding, management, and production for swine.

FIG. 11 is a schematic structural diagram showing a physical structure of an electronic device. As shown in FIG. 11, the electronic device may include a processor 1110, a communications interface 1120, a memory 1130, and a communication bus 1140. The processor 1110, the communications interface 1120, and the memory 1130 communicate with each other through the communication bus 1140. The processor 1110 may call logical instructions in the memory 1130 to perform the method for predicting net energy requirements for maintenance. The method includes: obtaining heart rate data of a target swine: and obtaining net energy requirements for maintenance of the target swine based on the heart rate data and a pre-trained net-energy-requirements prediction model, where the net-energy-requirements prediction model is a neural network model obtained by training based on capacity parameters.

In addition, the logical instructions in the memory 1130 may be stored in a computer-readable storage medium when implemented in a form of a software functional unit and sold or used as an independent product. Based on this understanding, the essence of the technical solution of the present disclosure or part of the technical solution that make contribution to the conventional technology or all or part of the technical solution may be embodied in a form of a software product, and the computer software product is stored in a storage medium, including several instructions for enabling a computer device (which may be a personal computer, a server, or a network device, etc.) to execute all or part of the steps of the method described in the various embodiments of the present disclosure. The storage medium described above includes various mediums which can store program codes, such as a USB flash disk, a mobile hard disk, a read-only memory (ROM), a random access memory (RAM), a disk, and an optical disc.

In another aspect, a computer program product is further provided according to the present disclosure. The computer program product includes a computer program that may be stored on a non-transient computer-readable storage medium. When the computer program is executed by a processor, the computer performs the above method for predicting net energy requirements for maintenance. The method includes: obtaining heart rate data of a target swine; and obtaining net energy requirements for maintenance of the target swine based on the heart rate data and a pre-trained net-energy-requirements prediction model, where the net-energy-requirements prediction model is a neural network model obtained by training based on capacity parameters.

In another aspect, a non-transient computer-readable storage medium storing a computer program thereon is further provided according to the present disclosure. The computer program, when being executed by a processor, implements the method for predicting net energy requirements for maintenance. The method includes: obtaining heart rate data of a target swine: and obtaining net energy requirements for maintenance of the target swine based on the heart rate data and a pre-trained net-energy-requirements prediction model, where the net-energy-requirements prediction model is a neural network model obtained by training based on capacity parameters.

Unless otherwise specified, the experimental methods used in the embodiments of the present disclosure are conventional methods and may be performed in accordance with the techniques or conditions described in literatures or product specifications in the art. Unless stated otherwise, all instruments, materials and reagents can be purchased through regular commercial channels. The device embodiments described above are merely illustrative. Units described as separate components may be or may not be physically separated. Components shown as units may be or may not be physical units, that is, the components may be arranged in one place or may be distributed in multiple network units. A part of or all the modules may be selected according to actual needs to achieve the objectives of the solutions of the embodiments. Those skilled in the art can understand and implement the present disclosure without any creative work.

Through the foregoing descriptions of the embodiments, it is clear to those skilled in the art that those embodiments may be implemented by means of software and a necessary universal hardware platform, and apparently may be implemented by means of hardware. Based on this understanding, the essence of the above technical solutions or the parts contributing to the conventional technology of the above technical solutions may be implemented as a software product. The software product may be stored in a computer-readable storage medium, such as an ROM/RAM, a diskette and an optical disk. The software product includes multiple instructions for enabling a computer device (such as a personal computer, a server, or a network device) to perform the method for predicting net energy requirements for maintenance described in the embodiments or in parts of the embodiments.

Finally, it should be noted that the above embodiments are merely provided for describing the technical solutions of the present disclosure, but are not intended to limit the present disclosure. Although the present disclosure is described in detail with reference to the foregoing embodiments, those skilled in the art should understand that, they can still modify technical solutions described in the foregoing embodiments, or make equivalent substitutions to a part or all of the technical features. The modifications or substitutions do not enable the essence of corresponding technical solutions to depart from the spirit and scope of the technical solutions in the embodiments of present disclosure.

1. A method for predicting net energy requirements for maintenance, comprising:
obtaining heart rate data of a target swine; and
obtaining net energy requirements for maintenance of the target swine based on the heart rate data and a pre-trained net-energy-requirements prediction model, wherein the net-energy-requirements prediction model is a neural network model obtained by training based on capacity parameters.

2. The method for predicting net energy requirements for maintenance according to claim 1, wherein a process of training the net-energy-requirements prediction model comprises:
obtaining heart rate data of several sample swine to form a first data set, wherein the heart rate data comprises time information;
obtaining net-energy-requirements-for-maintenance data of the several sample swine at corresponding time of the time information, to form a second data set;
establishing a training data set based on the first data set and the second data set;
obtaining the capacity parameters based on the training data set, a preset data curve fitting approach and a preset parameter estimation algorithm; and
training with respect to the capacity parameters based on the training data set to obtain the net-energy-requirements prediction model,
wherein the data curve fitting approach is to perform curve fitting on the training data set based on a nonlinear logical regression function.

3. The method for predicting net energy requirements for maintenance according to claim 2, wherein the obtaining the capacity parameters based on the training data set, the preset data curve fitting approach and the preset parameter estimation algorithm comprises:
performing curve fitting based on a nonlinear logical regression function, by taking the heart rate data in the training data set as input and taking the net-energy-requirements-for-maintenance data, corresponding to the heart rate data, in the training data set as output, to obtain a curve fitting function; and
performing inverse estimation of parameters based on the preset parameter estimation algorithm and the curve fitting function to obtain the capacity parameters.

4. The method for predicting net energy requirements for maintenance according to claim 3, wherein the curve fitting function is expressed as:
$${NEm}_{ij} = g ( {Φ}_{i} , {HR}_{ij} ) + {ε}_{ij}$$
wherein i represents a serial number of a sample swine, j represents a serial number of data, HRᵢⱼ represents heart rate data of a sample swine, NEmᵢⱼ represents net energy requirements for maintenance of a sample swine at corresponding time, Φᵢ represents capacity parameters, and εᵢⱼ represents a random effect error.

5. The method for predicting net energy requirements for maintenance according to claim 3, wherein the parameter estimation algorithm comprises any one or more of expectation-maximization algorithm, Newton's iteration algorithm and gradient descent algorithm.

6. The method for predicting net energy requirements for maintenance according to claim 2, wherein the process of training the net-energy-requirements prediction model further comprises:
establishing a test data set based on the first data set and the second data set, and recording the net-energy-requirements-for-maintenance data in the test data set as actual values of the net energy requirements for maintenance;
inputting the heart rate data in the test data set to the net-energy-requirements prediction model to obtain predicted values of the net energy requirements for maintenance;
analyzing correlation between the predicted values of the net energy requirements for maintenance and the actual values of the net energy requirements for maintenance; and
verifying the net-energy-requirements prediction model based on the correlation.

7. The method for predicting net energy requirements for maintenance according to claim 6, wherein the verifying the net-energy-requirements prediction model based on the correlation comprises:
obtaining distribution of prediction weighted residuals based on the correlation; and
verifying the net-energy-requirements prediction model based on the distribution of the prediction weighted residuals.

8. (canceled)

9. An electronic device, comprising:
a processor; and
a memory storing a computer program executable by the processor, wherein the processor is configured to execute the program to implement:
obtaining heart rate data of a target swine; and
obtaining net energy requirements for maintenance of the target swine based on the heart rate data and a pre-trained net-energy-requirements prediction model, wherein the net-energy-requirements prediction model is a neural network model obtained by training based on capacity parameters.

10. A non-transient computer-readable storage medium storing a computer program thereon, wherein the computer program is configured to, when being executed by a processor, implement:
obtaining heart rate data of a target swine; and
obtaining net energy requirements for maintenance of the target swine based on the heart rate data and a pre-trained net-energy-requirements prediction model, wherein the net-energy-requirements prediction model is a neural network model obtained by training based on capacity parameters.

11. The electronic device according to claim 9, wherein the processor is further configured to implement:
obtaining heart rate data of several sample swine to form a first data set, wherein the heart rate data comprises time information;
obtaining net-energy-requirements-for-maintenance data of the several sample swine at corresponding time of the time information, to form a second data set;
establishing a training data set based on the first data set and the second data set;
obtaining the capacity parameters based on the training data set, a preset data curve fitting approach and a preset parameter estimation algorithm; and
training with respect to the capacity parameters based on the training data set to obtain the net-energy-requirements prediction model,
wherein the data curve fitting approach is to perform curve fitting on the training data set based on a nonlinear logical regression function.

12. The electronic device according to claim 11, wherein the processor is further configured to implement:
performing curve fitting based on a nonlinear logical regression function, by taking the heart rate data in the training data set as input and taking the net-energy-requirements-for-maintenance data, corresponding to the heart rate data, in the training data set as output, to obtain a curve fitting function; and
performing inverse estimation of parameters based on the preset parameter estimation algorithm and the curve fitting function to obtain the capacity parameters.

13. The electronic device according to claim 12, wherein the curve fitting function is expressed as:
$${NEm}_{ij} = g ( {Φ}_{i} , {HR}_{ij} ) + {ε}_{ij}$$
wherein i represents a serial number of a sample swine, j represents a serial number of data, HRᵢⱼ represents heart rate data of a sample swine, NEmᵢⱼ represents net energy requirements for maintenance of a sample swine at corresponding time, Φᵢ represents capacity parameters, and εᵢⱼ represents a random effect error.

14. The electronic device according to claim 12, wherein the parameter estimation algorithm comprises any one or more of expectation-maximization algorithm, Newton's iteration algorithm and gradient descent algorithm.

15. The electronic device according to claim 11, wherein the processor is further configured to implement:
establishing a test data set based on the first data set and the second data set, and recording the net-energy-requirements-for-maintenance data in the test data set as actual values of the net energy requirements for maintenance;
inputting the heart rate data in the test data set to the net-energy-requirements prediction model to obtain predicted values of the net energy requirements for maintenance;
analyzing correlation between the predicted values of the net energy requirements for maintenance and the actual values of the net energy requirements for maintenance; and
verifying the net-energy-requirements prediction model based on the correlation.

16. The electronic device according to claim 15, wherein the processor is further configured to implement:
obtaining distribution of prediction weighted residuals based on the correlation; and
verifying the net-energy-requirements prediction model based on the distribution of the prediction weighted residuals.
